(19) United States
(12) Patent Application Publication
(10) Pub. No.: US 2006/0282056 A1
(43) Pub. Date: Dec. 14, 2006

(54) Diaper with integral disposal bag

(76) Inventor: Michael L. McDonald, Columbia, MD (US)

Correspondence Address: William S. Ramsey, 5253 Even Star Place, Columbia, MD, 21044, US

(21) Appl. No.: 11/152,801

(22) Filed: Jun. 14, 2005

(51) Int. Cl. A61F 13/15

(52) U.S. Cl. 604/385.13; 604/385.06

(57) ABSTRACT

An exemplary embodiment providing one or more improvements includes a fluid-impermeable bag attached to or integral with a disposable diaper, the bag having closure elements on the outside of the front and back panels of the uninverted bag. After use, the bag is inverted over the soiled diaper and the closure elements interact, thereby closing and sealing the soiled diaper inside the bag. In some embodiments the bag is covered by a removable fabric element, such as a gauze panel, which may be decorated.

### CROSS-REFERENCE(S)

Not Applicable.

### STATEMENT REGARDING FEDERALLY SPONSORED RESEARCH OR DEVELOPMENT

Not Applicable. cl REFERENCE TO A “MICROFICHE APPENDIX”

Not Applicable.

### DESCRIPTION OF RELATED ART INCLUDING INFORMATION DISCLOSED UNDER 37 CFR 1.97AND 37 CFR 1.98

### BACKGROUND

U.S. Pat. No. 3,865,110 discloses a disposable diaper with a removably attached bag. In use, one end of the bag is detached from the diaper, the soiled diaper is folded, and the bag is inverted over to enclose the soiled diaper.

U.S. Pat. No. 3,877,432 discloses a disposable diaper with associated disposal bag formed of two sheets of attached fluid-impervious back sheet. The used diaper is enclosed in the disposal bag by turning the back sheet inside out.

U.S. Pat. No. 4,430,087 discloses a disposable diaper with a folded bag attached at one end between the absorbent pad and backing. The bag is inverted over the soiled diaper. The bag containing the soiled diaper is sealed with an adhesive tab before disposal.

U.S. Pat. No. 4,923,455 discloses a disposable diaper with an integral envelope into which the soiled diaper is placed. The envelope is sealed with a resealable adhesive.

U.S. Pat. No. 4,964,859 discloses a disposable diaper with a folded integral changing pad. The pad has a drawstring which is used to close the pad as a bag about the soiled diaper.

U.S. Pat. No. 4,493,713 discloses disposable diaper with a detachable bag. The soiled diaper is placed in the bag which is sealed with a fastening strip.

U.S. Pat. No. 5,071,414 discloses a disposable diaper with an extra layer to the backing layer which forms a pocket at one end. In use, the soiled diaper is rolled up to the end with the pocket, ears on the sides of the diaper are extended over the roll and secured by adhesive strips, and the pocket is inverted over the rolled diaper, enclosing it.

U.S. Pat. No. 5,304,158 discloses a disposable diaper with an attached pouch containing a changing pad. The soiled diaper is wrapped in the pad which is placed in the pouch.

U.S. Pat. No. 6,454,748 discloses a diaper with a pocket. The pocket contains baby-changing related objects. The pocket has adhesive or other closure devices on the interior surface of the external layer for closing the pocket after the soiled diaper is rolled up and turned into the pocket.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

Embodiments include disposable diapers comprised of a fluid impervious backing sheet, fluid absorbent material, and a bag for enclosing the diaper for disposal, wherein the improvement comprises attachment of the bag to the impervious backing sheet with the bag opening adjacent to one end of the diaper, the bag capable of inversion with enclosure of the diaper, and a two element interactive closure on the outside of the uninverted bag,

Embodiments include a removable fabric cover panel which covers the attached bag. In embodiments the fabric cover, bag, and fluid impervious sheet is decorated with designs and indicia which may be personalized. In embodiments the bag is scented to mask offensive odors. In embodiments the bag contains a hand sanitizer or wipe for use in hand cleaning.

The following embodiments and aspects thereof are described and illustrated in conjunction with systems, tool and methods which are meant to be exemplary and illustrative, not limiting in scope. In various embodiments, one or more of the above-described problems have been reduced or eliminated, while other embodiments are directed to other improvements.

In addition to the exemplary aspects and embodiments described above, further aspects and embodiments will become apparent by reference to the drawings and by study of the following descriptions.

### BRIEF DESCRIPTION OF THE DRAWINGS

FIG. 1 is a plan view of the back of a first embodiment diaper.

FIG. 2 is a cross section view of a first embodiment diaper taken at arrow 2 of FIG. 1.

FIG. 3 is a plan view of the back of a second embodiment diaper.

FIG. 4 is a cross section view of a second embodiment diaper taken at arrow 3 of FIG. 3.

FIG. 5 is a cross sectional view of a closed bag with projecting ridge closure elements.

FIG. 6 is a cross sectional view of a closed bag with projecting ridge and slider closure elements.

FIG. 7 is a cross sectional view of a closed bag with male and female closure elements.

FIG. 8 is a cross sectional view of a closed bag with hook and loop closure elements.

FIG. 9 is a cross sectional view of a closed bag with adhesive closure elements.

FIG. 10 is a cross sectional view of a closed bag with self adhesive closure elements.

FIG. 11 is a flow chart of the process of bagging a first embodiment diaper.

FIG. 12 is a flow chart of the process of bagging a second embodiment diaper.

### DETAILED DESCRIPTION

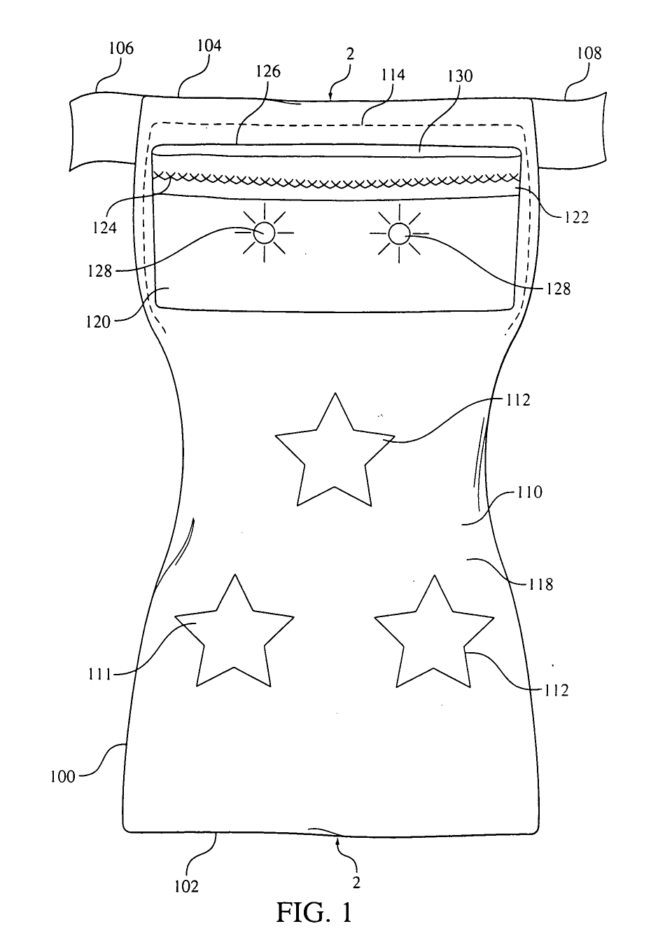

FIG. 1 is a plan view of the back of a first embodiment diaper 100. A fluid impermeable backing sheet 110 has a top end 104 and a bottom end 102. A left tab 106 and right tab 108 are adhesive coated tabs which are used to fasten the top end 104 to the bottom end 102 when the diaper is in use. A graphic 111 comprising a design or personalized indicia may appear on the backing sheet 110. A bag 120 is folded and attached near the top end 104 of the backing sheet 110. The bag opening 130 is near the top end 104. Visible in FIG. 1 are the front panel 122 of the bag and the back panel 126 of the bag. The front panel closure element 124 is visible in FIG. 1. A graphic 111 comprising a design or personalized indicia may appear on the front panel 124. The backing sheet 110 is overlaid by a cover 118 depicted by diagonal lines in FIG. 1. A U-shaped weakened area 114 of the cover 118, such as cuts in a dashed line, allows tearing of the cover from the bag when it is desired to bag the diaper. A graphic 112 comprising a design or personalized indicia may appear on the cover 118.

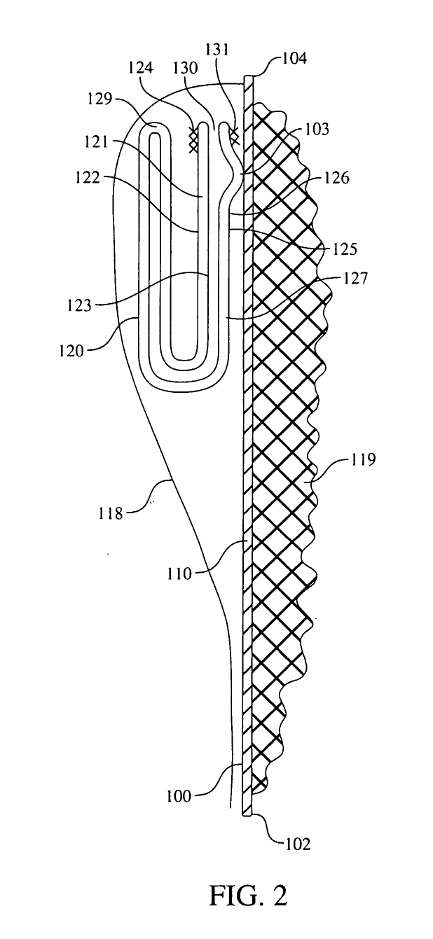

FIG. 2 is a cross section view of a first embodiment diaper 100 taken at arrow 2 of FIG. 1. Visible in FIG. 2 is a fluid impermeable sheet 110 with the diaper top 104 and bottom 102. Absorbent material 119 which absorbs body wastes is attached to the sheet 110. The bag 120 is comprised of outer panel 122 and inner panel 126 which are joined at the bag bottom 129 and an opening 130 at the top of the bag. Outer panel 122 has an exterior side 121 and an interior side 123. Inner panel 126 has an exterior side 125 and an interior side 127. An outer panel connector element 124 is located at the exterior side 121 of the outer panel 122. An inner panel connector element 131 is located at the exterior side 125 of the inner panel 126. When the bag 120 is inverted the outer panel connector element 124 interacts with the inner panel connector element 131 and secures the inverted bag in a closed condition. The inner panel 126 is connected and secured to the fluid impermeable sheet 110 at connection site 103 which secures the bag 120 to the sheet 110 near the diaper top 104. In the first embodiment diaper 100 the bag 120 is folded at least once. The fluid impermeable sheet 110 and the bag 120 is enclosed by a cover 118.

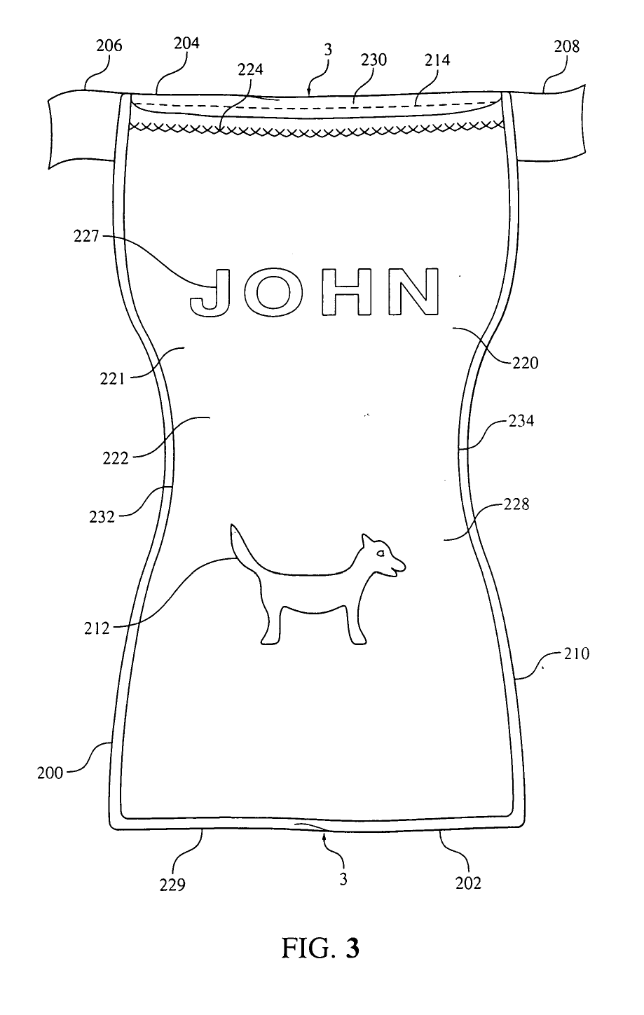

FIG. 3 is a plan view of the back of a second embodiment diaper 200. A fluid impermeable backing sheet 210 has a top end 204 and a bottom end 202. A left tab 206 and right tab 208 are adhesive coated tabs which are used to fasten the top end 204 to the bottom end 202 when the diaper is in use. A bag 220 is comprised of a bag front panel 222 and the fluid impermeable backing sheet 210 which plays the role of the back bag panel of the first embodiment diaper. The front panel 222 is connected to the backing sheet 210 at the left side 232, right side 234, and bottom 229. The bag opening 230 is near the top end 204. The front panel closure element 224 is visible in FIG. 3. A graphic 212 comprising a design or personalized indicia may appear on the front panel 222. The front panel 222 is overlaid by a cover 228, depicted by diagonal lines in FIG. 3. A weakened area 214 of the cover material, such as cuts in a dashed line, allows tearing of the cover and allows access to the opening 230 of the bag when it is desired to bag the diaper. A graphic 227 comprising a design or personalized indicia may appear on the cover 228.

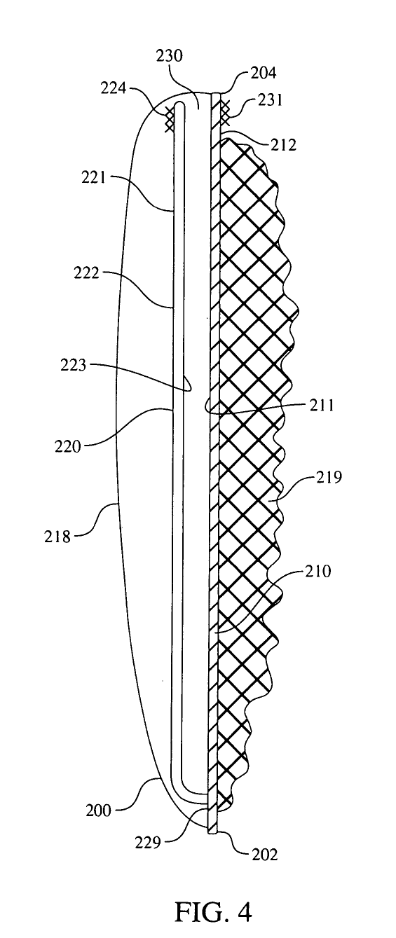

FIG. 4 is a cross section view of the second embodiment diaper 200 taken at arrows 3 of FIG. 3. Visible in FIG. 4 is a fluid impermeable sheet 210 (also bag inner panel) with the diaper top 204 and bottom 202. Absorbent material 219 which absorbs body wastes is attached to the exterior side 212 of the fluid impermeable sheet 210. The bag 220 is comprised of outer panel 222 and inner panel 210 (also fluid impermeable sheet) which are joined at the bag bottom 229 and an opening 330 at the top of the bag. In the second embodiment the fluid impermeable sheet 210 is also the bag inner panel 210. Outer panel 222 has an exterior side 221 and an interior side 223. Inner panel or fluid impermeable sheet 210 has an exterior side 212 and an interior side 211. An outer panel connector element 224 is located at the exterior side 221 of the outer panel 222. An inner panel connector element 231 is located at the exterior side 212 of the inner panel or fluid impermeable sheet 210. When the bag 220 is inverted the outer panel connector element 224 interacts with the inner panel connector element 231 and secures the inverted bag in a closed condition. In the second embodiment diaper 200 the bag 220 is unfolded and extends approximately from the top 204 to the bottom 202 of the diaper. The fluid impermeable sheet 210 and the bag 220 is enclosed by a cover 218.

Any diaper having a fluid-impermeable backing sheet can be used with this invention. Suitable diapers include HUGGIES. HUGGIES is a trademark for diapers owned by Kimberly-Clark, Dallas Tex. Suitable diapers also include PAMPERS. PAMPERS is a trademark for diapers owned by The Procter & Gamble Company, Cincinnati, Ohio.

Any suitable fabric material capable of concealing the bag yet allowing easy removal may be used for the cover. Suitable materials include woven or unwoven plastic, gauze, paper, and cotton fabric.

Any suitable two element closure system which provides for secure closure of an inverted bag containing a soiled diaper may be used. The following embodiments illustrate a variety of suitable closures. These elements are illustrated with the bag elements of the first embodiment, but they can be used with any embodiment.

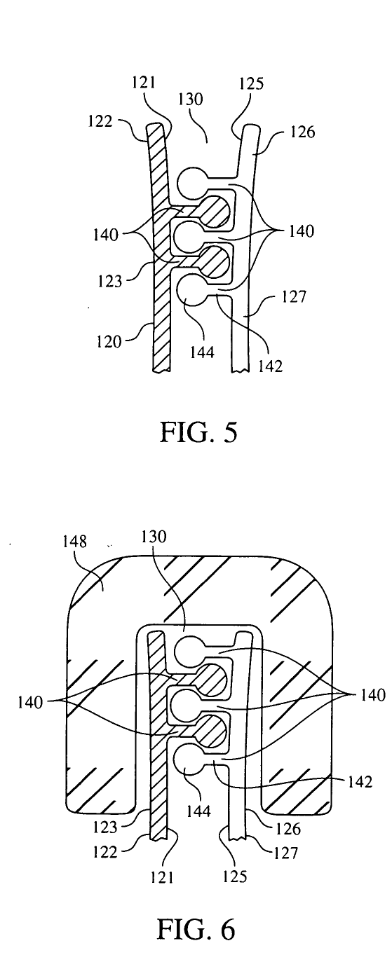

FIG. 5 is a cross section view of an embodiment with an inverted bag closed with parallel projecting ridge closure. Visible in FIG. 5 is the outer panel 122 of the bag 120 with the interior side 123 and exterior side 121. Also visible is the inner panel 126 with the interior side 127 and exterior side 125. Two or more parallel projecting ridges 140 are spaced apart and arrayed near the bag top 130 attached to the exterior side 121 of the outer panel 120 and the exterior side 125 of the inner panel 126. Each ridge 140 is comprised of an elongated web 142 and an expanded head 144. In use the ridges are pressed together and the expanded heads 144 interact and hold the closure elements in a closed position. Suitable bags include ZIPLOC brand bags. ZIPLOC is a trademark for bags owned by S.C. Johnson & Son, Inc., Racine, Wis.

FIG. 6 is a cross sectional view of an embodiment in which the closed lo 0 inverted bag with parallel projecting ridge closure elements are urged together by a slider 148 which is manually drawn along the fasteners from one end to another. The other elements of FIG. 6 are the same as in FIG. 5. The parallel projecting ridge closure elements are attached about the circumference of the outside of the uninverted bag with the slider 148 at one intersection between the front and back panels of the bag. Suitable bags include HEFTY bags with ONEZIP sliders, also termed EASY GRIP sliders. HEFTY, ONEZIP, and EASY GRIP are trademarks owned by Pactiv Corporation, Lake Forest, Ill. for sliders sold as a component part of multi-purpose household bags, plastic bags for multipurpose household use, namely, closeable storage and freezer bags, and a ribbed slider sold as a component of plastic bags for food storage and general purpose use, respectively.

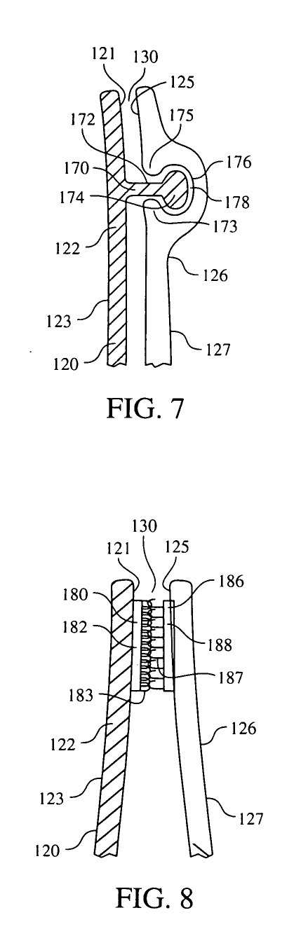

FIG. 7 is an embodiment in which the bag is closed using male and female element fasteners which interact to close the opening of the bag. Visible in FIG. 7 is the outer panel 122 of the bag 120 with the interior side 123 and exterior side 121. Also visible is the inner panel 126 with the interior side 127 and exterior side 125. The male closure element is an elongated ridge 170 comprising a web 172 and an expanded head 174. The female closure element 178 is comprised of an elongated groove 176 with adjoining lips 173 and 175. The fastener elements are attached about the circumference of the outside of the uninverted bag at the top 130 of the bag. The inverted bag is closed by pressing together the male 170 and female 178 elements of the fasteners. In the closed orientation, the expanded head 174 is retained in the groove 176 by the lips 173 and 175.

FIG. 8 is an embodiment in which the bag is closed using hook and loop fasteners elements attached to the circumference of the outside of the uninverted bag. Visible in FIG. 8 is the outer panel 122 of the bag 120 with the interior side 123 and exterior side 121. Also visible is the inner panel 126 with the interior side 127 and exterior side 125. The loop element 180 of the fastener is comprised of a attachment strip 182 to which a multiplicity of loops 183 is connected. The hook element 186 is comprised of an attachment strip 188 to which a multiplicity of loops 187 is connected. The fastener elements are attached about the circumference of the outside of the uninverted bag at the top of the bag 130. Suitable fasteners include VELCRO hook and loop fasteners. VELCRO is a trademark for hook and loop fasteners owned by Velcro Industries B.V., a Dutch Company, and may be obtained from Velcro USA Inc., Manchester, N.H.

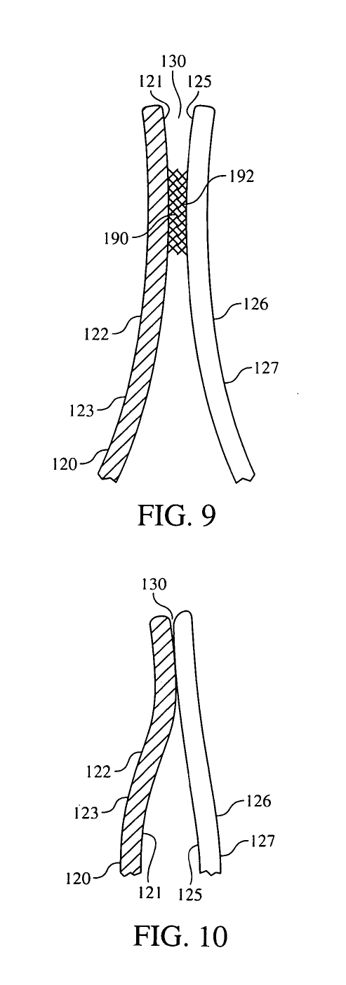

FIG. 9 is another embodiment in which a strip of pressure sensitive adhesive is applied to both the front and rear panels of the uninverted bag. Visible in FIG. 9 is the outer panel 122 of the bag 120 with the interior side 123 and exterior side 121. Also visible is the inner panel 126 with the interior side 127 and exterior side 125. A strip of pressure sensitive adhesive 190 is spread on the exterior side 121 of the outer panel 122 and another strip of pressure sensitive adhesive 192 is spread on the exterior side 125 of the inner panel 126 near the top 130 of the bag 120. These adhesives use polymers, such as acrylics, rubbers, and polyurethanes, together with plasticizers and tackifying resins to form a permanently tacky or sticky adhesive. A separate release strip of paper or plastic covers the adhesive strips and protects the strips until the release strip is removed before the bag is inverted. The strips of pressure sensitive adhesive on the front and rear panels of the inverted bag is then pressed together, thereby sealing the bag. Suitable materials include pressure sensitive adhesives obtainable from Adchem Manufacturing, Riverhead, N.Y.

FIG. 10 is an embodiment in which the bag is manufactured of a plastic film having one surface which is adhesive when pressed against a similar surface. Visible in FIG. 10 is the outer panel 122 of the bag 120 with the interior side 123 and exterior side 121. Also visible is the inner panel 126 with the interior side 127 and exterior side 125. The self adhesive surface is on the exterior side 123 of the outer panel 122 and on the exterior side 121 of the inner panel 127 of the uninverted bag near the top 130 of the bag 120. One element of the closure of this embodiment is the exterior surface of the outer panel of the bag and the other element is the exterior side of the inner panel of the bag. When inverted over the soiled diaper, the self adhesive surfaces about the circumference of the inverted bag are manually pressed together, thereby sealing the soiled diaper into the bag for disposal. Any suitable strong film material having at lease one surface self adhesive can be used in this embodiment diaper. Suitable materials include films obtainable from The Glad Products Company, Oakland Calif. using the trademarks PRESS'N SEAL, PRESS'N SEAL FREEZER, and GRIPTEX. PRESS'N SEAL and PRESS'N SEAL FREEZER are trademarks for plastic wrap; plastic bags owned by The Glad Products Company, Oakland Calif. GRIPTEX is a trademark for general purpose plastic bags and plastic wrap owned by Procter & Gamble Company, Cincinnati, Ohio and licensed to The Glad Products Company, Oakland, Calif.

Although the closure elements illustrated in FIGS. 5-10 are illustrated in connection with the first embodiment diaper, the closure elements may be used with the second and any other embodiment.

The interior surfaces of the bag of embodiments are scented to eliminate or suppress unpleasant odors associated with soiled diapers.

The bags of embodiments contain moistened tissues for use in sanitizing and cleaning associated with the changing of a diaper. The used wipes are placed in the inverted bag for disposal after use.

Diapers of embodiments may be used by infants, babies, toddlers, or adults.

The material of the bag in embodiments is opaque thereby concealing the contents when the bag contains a soiled diaper.

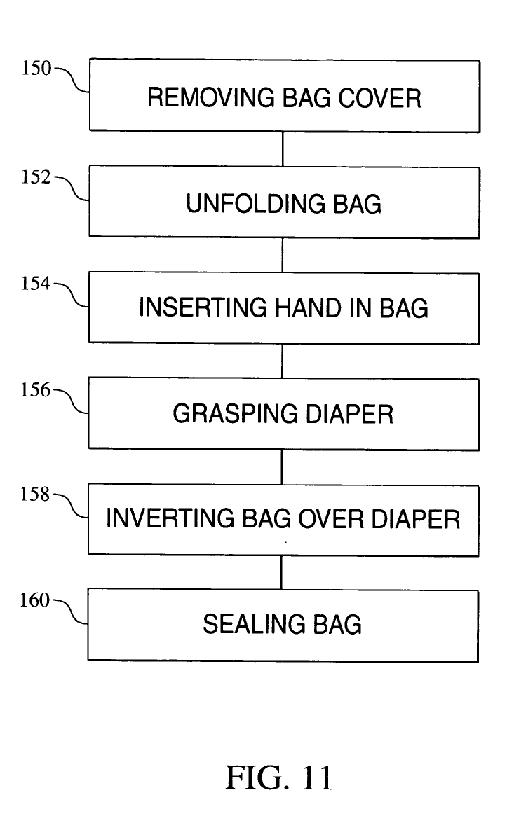

FIG. 11 is a flow chart of the process of bagging a first embodiment diaper. The first step 150 is to remove the bag cover by tearing at the weakened areas. The next step is unfolding the bag 152. The user inserts one hand into the bag 154, with the palm toward the back side of the diaper. The user then grasps the diaper 156 with the inserted hand and, optionally, folds the diaper by closing the fingers. The user the inverts the bag over the diaper 158 with the user's other hand. The user then seals the bag 160 using the closure elements of the now inverted bag.

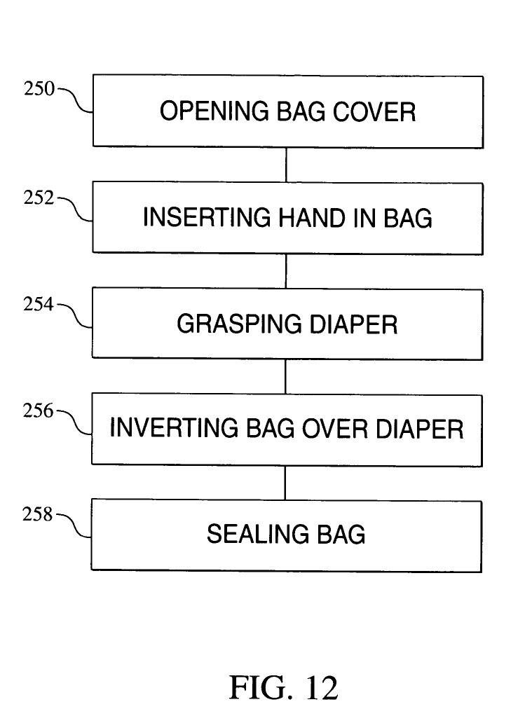

FIG. 12 is a flow chart of the process of bagging a second embodiment diaper. The first step 250 is to open the bag cover at the weakened area. The user inserts one hand into the bag 252, with the palm toward the back side of the diaper. The user then grasps the diaper 254 with the inserted hand and, optionally, folds the diaper by closing the fingers. The user the inverts the bag over the diaper 256 with the user's other hand. The user then seals the bag 258 using the closure elements of the now inverted bag.

While a number of exemplary aspects and embodiments have been discussed above, those of skill in the art will recognize certain modifications, permutations, additions and subcombinations thereof. It is therefore intended that the following appended claims and claims hereafter introduced are interpreted to include all such modifications, permutations, additions and sub-combinations as are within their true spirit and scope.

I claim:

1. A disposable diaper comprised of a fluid impervious backing sheet, a fluid absorbent material, and a bag for enclosing the diaper for disposal, wherein the improvement comprises
attachment of the bag to the impervious backing sheet with the bag opening adjacent to one end of the diaper,
the bag capable of inversion with enclosure of the diaper, and
a two element interactive closure on the outside of the uninverted bag,

2. The improvement of claim 1 wherein the closure elements are arrayed about the circumference of the opening of the bag.

3. The improvement of claim 1 wherein one panel of the bag is the fluid impermeable backing sheet.

4. The improvement of claim 1 wherein the closure elements are male and female elements which interact when the bag is closed.

5. The improvement of claim 1 wherein the closure elements are spaced apart projections which interact when the bag is closed.

6. The improvement of claim 5 further comprising a slider which forces the projections into interaction when the bag is closed.

7. The improvement of claim 1 wherein the closure elements are hook and loop closure elements which interact when the bag is closed.

8. The improvement of claim 1 wherein the closure elements are adhesive elements which interact when the bag is closed.

9. The improvement of claim 1 wherein the closure elements are self adhesive bag panels which adhere on contact.

10. The improvement of claim 1 further comprising a removable panel which covers the bag.

11. The improvement of claim 10 wherein the removable panel has a weakened region to allow easy removal of the panel.

12. The improvement of claim 11 wherein the removable panel is made of gauze material.

13. The improvement of claim 1 further comprising a scent in the bag.

14. The improvement of claim 1 further comprising moistened tissues contained in the bag.

15. The improvement of claim 1 further comprising designs or indicia on the bag, cover or backing sheet which may customized by the user.

16. The process of bagging a soiled diaper having an attached bag and a bag cover comprising the steps:
a. removing the bag cover,
b. unfolding the bag,
c. inserting one hand into the bag, with the palm toward the back side of the diaper,
d. grasping the diaper with the inserted hand,
e. inverting the bag over the diaper using the other hand, and
f. sealing the bag using the closure elements.

17. The process of claim 16 further comprising the step after step d. and before step e.:
g′. folding the grasped diaper,

18. The process of bagging a soiled diaper having an integral bag and a bag cover comprising the steps:
a. removing the bag cover,
b. inserting one hand into the bag, with the palm toward the back side of the diaper,
c. grasping the diaper with the inserted hand,
d. inverting the bag over the diaper using the other hand, and
e. sealing the bag using the closure elements.

19. The process of claim 18 further comprising the step after step c. and before step d.:
c′. folding the grasped diaper,
